(19) United States
(12) Patent Application Publication
(10) Pub. No.: US 2024/0299415 A1
(43) Pub. Date: Sep. 12, 2024

(54) TREATMENT METHOD FOR INTERSTITIAL PNEUMONIA

(71) Applicant: Shenzhen Evergreen Therapeutics Co., Ltd, Shenzhen (CN)

(72) Inventors: Tao Tom Du, Shenzhen (CN); Tao Hu, Shenzhen (CN); Xin Du, Shenzhen (CN)

(21) Appl. No.: 18/549,968

(22) PCT Filed: Dec. 1, 2021
(86) PCT No.: PCT/CN2021/134828

§ 371 Date: Sep. 11, 2023

(30) Foreign Application Priority Data
Dec. 1, 2020 (CN) 202011388458.0
Nov. 29, 2021 (CN) 202111428491.6

(51) Int. Cl. A61K 31/57; A61K 31/573; A61P 11/00

(52) U.S. Cl. CPC A61K 31/57; A61K 31/573; A61P 11/00

(57) ABSTRACT

A treatment method for interstitial pneumonia by means of hydroxyprogesterone caproate alone or in combination with other drugs, and corresponding pharmaceutical uses, and pharmaceutical compositions. Hydroxyprogesterone caproate can reduce pulmonary inflammation and fibrosis symptoms, reduce pulmonary collagen deposition, and reduce the levels of various inflammatory factors in bronchoalveolar lavage fluid and plasma. Hydroxyprogesterone caproate combined with dexamethasone can improve lipid metabolism abnormalities caused by dexamethasone treatment alone.

### CROSS REFERENCE TO RELATED APPLICATIONS

This application is the U.S. National Stage of PCT/CN2021/134828 filed Dec. 1, 2021, which claims priority to the Chinese patent application No. 202011388458.0, filed with the Patent Office of China on Dec. 1, 2020, with the title of “Treatment Method for Interstitial Pneumonia”, the entire contents of which are incorporated herein by reference; it also claims priority to the Chinese patent application No. 202111428491.6, filed with the Patent Office of China on Nov. 29, 2021, with the title of “Use of hydroxyprogesterone caproate in the preparation of drugs for treating interstitial pneumonia”.

### TECHNICAL FIELD OF THE INVENTION

The present application provides treatment methods of interstitial pneumonitis with hydroxyprogesterone caproate alone or in combination with other drugs, and corresponding pharmaceutical uses, pharmaceutical compositions, and the like.

### BACKGROUND OF THE INVENTION

Interstitial lung disease (ILD) is a heterogeneous group of diseases characterized by disorders of the cells within the alveolar structures and the matrix within the connective tissues, with the main clinical symptoms including dyspnea, hypoxemia, and decreased diffusion of carbon monoxide (DLCO). The broad definition of ILD includes almost all diseases that occur in the interstitium of the lungs with different etiologies, pathogenesis and lesions. Currently, there are nearly 200 known causes of ILD, including dust, infections, medications, inhalation of special gases, etc., but the specific etiology of ILD cannot be determined in more than 70% of ILD cases. Idiopathic interstitial pneumonia (IIP) is a group of unexplained ILD characterized by diffuse alveolitis and alveolar structural disturbances ultimately leading to pulmonary fibrosis. According to the 2002 classification of the American Thoracic Society (ATS) and the European Respiratory Society (ERS), IIP consists of the following 7 main types Subtypes: idiopathic pulmonary fibrosis (IPF), nonspecific interstitial pneumonia (NSIP), cryptogenic opportunistic pneumonia (COP), acute interstitial pneumonia (AIP), respiratory bronchiolitis with interstitial lung disease (RBILD), desquamative interstitial pneumonia (DIP), and lymphocytic interstitial pneumonia (LIP). Among them, IPF is the most common and representative disease in IIP, which has been reported to account for more than 60% of IIP morbidity. The interstitium of the lungs in patients with the disease will continue to produce fibrosis, which in turn leads to thickening of the lung tissues, resulting in irreversible loss of the ability of the lung tissues to exchange oxygen. IPF is a fatal respiratory disease, and the average patient's survival time is only 2-3 years after diagnosis.

Neither the etiology nor the pathogenesis of IIP and IPF has been fully elucidated. In general, the persistence or recurrence of lung tissue injury leads to dysregulation of cytokines, chemokines, and growth factors, abnormal vascular regeneration, impaired fibrinolytic system, reduced extracellular matrix degradation, and enhanced oxidative stress, resulting in changes in the alveolar microenvironment that inhibit the host inflammatory response. However, the altered alveolar microenvironment may also lead to dysregulated repair and abnormal tissue remodeling, resulting in failure of re-epithelialization of alveolar epithelial cells, recruitment and activation of fibroblasts or myofibroblasts, and ultimately progressive fibrosis. Due to the unclear etiology, the current treatments for IIP are very limited. In addition to symptomatic treatment, only glucocorticosteroids and immunosuppressive drugs have been shown to be effective, and the therapeutic effect depends on different subtypes of IIP. The inflammation during the pathogenesis of IPF is relatively mild compared to other interstitial pneumonias, while fibrosis predominates, so the therapeutic effects of glucocorticosteroids and immunosuppressive drugs are both very limited. Therefore, the therapeutic effects of glucocorticoids and immunosuppressants are very limited. In recent years, studies on the abnormal proliferation and function of fibroblasts, apoptosis of alveolar epithelial cells, and the TGF-β signaling pathway in pulmonary fibrosis have also shown that high-dose glucocorticoids are no longer recommended for the treatment of IPF, but there is still no truly effective treatment. Non-IPF IIP treatment strategies are different, such as COP, DIP and RBILD have a better response to glucocorticoids, and the prognosis is not the same as IPF. LIP, on the other hand, needs to be supplemented with certain immunosuppressants or even cytotoxic drugs to achieve a better therapeutic effect. Therefore, glucocorticoid therapy for IIP should also be subtype-specific and differentiated.

In addition to the lack of therapeutic efficacy for IIP, especially IFP, glucocorticosteroids have the following problems: 1) Resistance. Glucocorticoid resistance or insensitivity is a difficult problem to manage in diseases treated with glucocorticoids, characterized by failure to respond to adequate doses of corticosteroids, the need for very high doses of therapy, or an initial response to corticosteroids with a rapid relapse when the drug is stopped or the dose is tapered. The molecular mechanisms of glucocorticoid resistance include reduced expression of the glucocorticoid receptor, defects and translocations in binding to the glucocorticoid receptor, genetic susceptibility, and activation of inflammatory signaling pathways, which have been reported to prevent effective glucocorticoid therapy in 20-40% of patients. 2) Side effects. Due to the wide range of pharmacological activities of glucocorticoids, long-term administration of large doses will inevitably cause side effects and complications, and in severe cases, may even lead to the death of the patient. Common clinical side effects include secondary infections, digestive symptoms such as gastric ulcers, cardiovascular system-related symptoms such as hypertension, edema, elevated blood glucose, Cushing's syndrome such as centripetal obesity, full-moon face, neuropsychiatric symptoms such as euphoria, insomnia, irritability and other mood changes, osteoarthritic complications such as osteoporosis, and ophthalmologic disorders such as glaucoma and cataracts, and so on. Therefore, how to increase the sensitivity of glucocorticoid drugs, reduce the dosage of glucocorticoid and attenuate its side effects are also urgent problems to be solved in clinical practice.

Given the current lack of effective clinical treatments for IIP, particularly IFP, and the problems of resistance and side effects of existing glucocorticoid treatments, there exists a great need in the field for drugs for the treatment of this disease.

### SUMMARY OF THE INVENTION

On the one hand, the present application provides the use of hydroxyprogesterone caproate in the preparation of a drug for the treatment of interstitial pneumonia.

Further, wherein hydroxyprogesterone caproate is the sole active ingredient in said drug.

On the other hand, the present application provides the use of hydroxyprogesterone caproate and glucocorticoids in the preparation of drugs for the treatment of interstitial pneumonia.

Further, wherein said glucocorticoid is dexamethasone.

Further, wherein the pharmaceutical dosage form is an injection, an oral formulation or a topical formulation; preferably an injection, an oral formulation; particularly preferred is an injection.

Further, wherein said injections include injections, powders; oral preparations include tablets, capsules, oral liquids, throat or nasal sprays, and topical preparations include ointments, sprays, patches.

Further, wherein said treating interstitial pneumonia comprises reducing symptoms of inflammation and fibrosis in the lungs.

Further, wherein said treating interstitial pneumonia comprises reducing collagen deposition in the lungs.

Further, wherein said treating interstitial pneumonia comprises reducing an inflammatory cell count in bronchoalveolar lavage fluid.

Further, wherein the treatment of interstitial pneumonitis described therein comprises reducing hydroxyproline levels in lung tissue.

Further, wherein said treating interstitial pneumonia comprises reducing levels of a plurality of inflammatory factors in alveolar lavage fluid and plasma; preferably, said inflammatory factors comprise TNF-α, IL-1β, IFN-γ, IL-6, TGF-β1, IL-4, MCP-1/CCL2.

Further, wherein said treating interstitial pneumonitis comprises decreasing the levels of a plurality of pro-fibrotic and inflammatory cytokines and type I collagen in alveolar lavage fluid and/or plasma, and/or increasing the levels of anti-fibrotic factors; preferably, said pro-fibrotic factors and inflammatory cytokines are TNF-α, IL-1β, IL-6, MCP-1, PDGF, SMAD3, SMAD2, the MMP9, MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein the abnormalities of lipid metabolism during said treatment of interstitial pneumonitis are reduced.

Further, wherein said abnormalities of lipid metabolism comprise elevated levels of triglycerides and total cholesterol and/or reduced levels of leptin.

On the other hand, the present application provides methods of treating interstitial pneumonia, characterized in that said methods comprise administering hydroxyprogesterone caproate to a patient with interstitial pneumonia.

Further, wherein said method does not comprise the step of administering a glucocorticoid.

Further, wherein said method comprises the step of administering a glucocorticoid.

Further, wherein said glucocorticoid is dexamethasone.

Further, wherein the dosage form of hydroxyprogesterone caproate is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein the hydroxyprogesterone caproate dose is 30-100 mg/day, or 250-750 mg/week.

Further, wherein the daily dose of hydroxyprogesterone caproate is 20-80 mg/kg; preferably 40-80 mg/kg; more preferably 40 or 80 mg/kg.

Further, wherein the hydroxyprogesterone caproate administration regimen is administered 1-7 times per week, preferably 1-4 times per week, more preferably 1-2 times per week.

Further, wherein said treating interstitial pneumonia comprises reducing symptoms of inflammation and fibrosis in the lungs.

Further, wherein said treating interstitial pneumonia comprises reducing collagen deposition in the lungs.

Further, wherein said treating interstitial pneumonia comprises reducing an inflammatory cell count in bronchoalveolar lavage fluid.

Further, wherein said treating interstitial pneumonitis comprises reducing hydroxyproline levels in lung tissue.

Further, wherein said treating interstitial pneumonia comprises reducing levels of a plurality of inflammatory factors in alveolar lavage fluid and plasma; preferably, said inflammatory factors comprise TNF-α, IL-1β, IFN-γ, IL-6, TGF-β1, IL-4, MCP-1/CCL2.

Further, wherein said treating interstitial pneumonia comprises reducing levels of a plurality of pro-fibrotic factors and inflammatory cytokines and type I collagen in the alveolar lavage fluid and/or plasma, and/or increasing levels of anti-fibrotic factors; preferably, said pro-fibrotic factors and inflammatory cytokines are TNF-α, IL-1β, IL-6, MCP-1, PDGF, SMAD3, SMAD2. MMP9, MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein the abnormalities of lipid metabolism during said treatment of interstitial pneumonitis are reduced.

Further, wherein said abnormalities of lipid metabolism comprise elevated levels of triglycerides and total cholesterol and/or reduced levels of leptin.

On the other hand, the present application provides drugs for treating interstitial pneumonia, characterized in that said drugs comprise hydroxyprogesterone caproate.

Further, wherein hydroxyprogesterone caproate is the sole active ingredient in said drug.

Further, wherein said drug further comprises a glucocorticoid.

Further, wherein said glucocorticoid is dexamethasone.

Further, wherein said pharmaceutical dosage form is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein said drug is used to reduce symptoms of inflammation and fibrosis in the lungs.

Further, wherein said drug is used to reduce collagen deposition in the lungs.

Further, wherein said drug is used to reduce inflammatory cell counts in bronchoalveolar lavage fluid.

Further, wherein said drug is used to reduce hydroxyproline levels in lung tissue.

Further, wherein said drug is used to reduce levels of a plurality of inflammatory factors in alveolar lavage fluid and plasma; preferably, said inflammatory factors include TNF-α, IL-1β, IFN-γ, IL-6, TGF-β1, IL-4, MCP-1/CCL2.

Further, wherein said drug is used to reduce levels of a plurality of pro-fibrotic factors and inflammatory cytokines and type I collagen in the alveolar lavage fluid and/or plasma, and/or to increase levels of anti-fibrotic factors; preferably, said pro-fibrotic factors and inflammatory cytokines are TNF-α, IL-1β, IL-6, MCP-1, PDGF, SMAD3, SMAD2, MMP9, the MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein the abnormalities of lipid metabolism are reduced during said drug treatment of interstitial pneumonia.

Further, wherein said abnormalities of lipid metabolism comprise elevated levels of triglycerides and total cholesterol and/or reduced levels of leptin.

Further, wherein said drug hydroxyprogesterone caproate is dosed at 30-100 mg/day, or 250-750 mg/week, and/or the hydroxyprogesterone caproate administration regimen is administered 1-7 times per week, preferably 1-4 times per week, and more preferably 1-2 times per week.

Further, wherein said drug hydroxyprogesterone caproate is dosed at a daily dose of 20-80 mg/kg; preferably 40-80 mg/kg; more preferably 40 or 80 mg/kg.

On the other hand, the present application provides the use of hydroxyprogesterone caproate in the preparation of drugs for the treatment of pulmonary fibrosis.

Further, wherein hydroxyprogesterone caproate is the sole active ingredient in said drug.

On the other hand, the present application provides the use of hydroxyprogesterone caproate and glucocorticoids in the preparation of drugs for the treatment of pulmonary fibrosis.

Further, wherein said glucocorticoid is dexamethasone.

Further, wherein the pharmaceutical dosage form is an injection, an oral formulation or a topical formulation; preferably an injection, an oral formulation; particularly preferred is an injection.

Further, wherein said injections include injections, powders; oral preparations include tablets, capsules, oral liquids, throat or nasal sprays, and topical preparations include ointments, sprays, patches.

Further, wherein said treating pulmonary fibrosis comprises reducing symptoms of inflammation and fibrosis in the lungs.

Further, wherein said treating pulmonary fibrosis comprises reducing collagen deposition in the lungs.

Further, wherein said treating pulmonary fibrosis comprises reducing inflammatory cell counts in bronchoalveolar lavage fluid.

Further, wherein said treating pulmonary fibrosis comprises reducing hydroxyproline levels in lung tissue.

Further, wherein said treating pulmonary fibrosis comprises reducing levels of a plurality of inflammatory factors in alveolar lavage fluid and plasma; preferably, said inflammatory factors comprise TNF-α, IL-1β, IFN-γ, IL-6, TGF-β1, IL-4, MCP-1/CCL2.

Further, wherein said treating pulmonary fibrosis comprises decreasing the levels of a plurality of pro-fibrotic and inflammatory cytokines and type I collagen in alveolar lavage fluid and/or plasma, and/or increasing the levels of an anti-fibrotic factor; preferably said pro-fibrotic factors and inflammatory cytokines are TNF-alpha, IL-1β, IL-6, MCP-1, PDGF, SMAD3, SMAD2, the MMP9, MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein the abnormalities of lipid metabolism during said treatment of pulmonary fibrosis are reduced.

Further, wherein said abnormalities of lipid metabolism comprise elevated levels of triglycerides and total cholesterol and/or reduced levels of leptin.

On the other hand, the present application provides methods of treating pulmonary fibrosis, characterized in that said methods comprise administering hydroxyprogesterone caproate to a patient with pulmonary fibrosis.

Further, wherein said method does not comprise the step of administering a glucocorticoid.

Further, wherein said method comprises the step of administering a glucocorticoid.

Further, wherein said glucocorticoid is dexamethasone.

Further, wherein the dosage form of hydroxyprogesterone caproate is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein the dose of hydroxyprogesterone caproate is 30-100 mg/day, or 250-750 mg/week.

Further, wherein the daily dose of hydroxyprogesterone caproate is 20-80 mg/kg; preferably 40-80 mg/kg; more preferably 40 or 80 mg/kg.

Further, wherein the hydroxyprogesterone caproate administration regimen is administered 1-7 times per week, preferably 1-4 times per week, more preferably 1-2 times per week.

Further, wherein said treating pulmonary fibrosis comprises reducing symptoms of inflammation and fibrosis in the lungs.

Further, wherein said treating pulmonary fibrosis comprises reducing collagen deposition in the lungs.

Further, wherein said treating pulmonary fibrosis comprises reducing inflammatory cell counts in bronchoalveolar lavage fluid.

Further, wherein said treating pulmonary fibrosis comprises reducing hydroxyproline levels in lung tissue.

Further, wherein said treating pulmonary fibrosis comprises reducing levels of a plurality of inflammatory factors in alveolar lavage fluid and plasma; preferably, said inflammatory factors comprise TNF-α, IL-1β, IFN-γ, IL-6, TGF-β1, IL-4, MCP-1/CCL2.

Further, wherein said treating pulmonary fibrosis comprises reducing levels of a plurality of pro-fibrotic factors and inflammatory cytokines and type I collagen in alveolar lavage fluid and/or plasma, and/or increasing levels of anti-fibrotic factors; preferably, said pro-fibrotic factors and inflammatory cytokines are TNF-α, IL-1β, IL-6, MCP-1, PDGF, SMAD3, SMAD2. MMP9, MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein the abnormalities of lipid metabolism during said treatment of pulmonary fibrosis are reduced.

Further, wherein said abnormalities of lipid metabolism comprise elevated levels of triglycerides and total cholesterol and/or reduced levels of leptin.

On the other hand, the present application provides drugs for the treatment of pulmonary fibrosis, characterized in that said drugs comprise hydroxyprogesterone caproate.

Further, wherein hydroxyprogesterone caproate is the sole active ingredient in said drug.

Further, wherein said drug further comprises a glucocorticoid.

Further, wherein said glucocorticoid is dexamethasone.

Further, wherein said pharmaceutical dosage form is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein said drug is used to reduce symptoms of inflammation and fibrosis in the lungs.

Further, wherein said drug is used to reduce collagen deposition in the lungs.

Further, wherein said drug is used to reduce inflammatory cell counts in bronchoalveolar lavage fluid.

Further, wherein said drug is used to reduce hydroxyproline levels in lung tissue.

Further, wherein said drug is used to reduce levels of a plurality of inflammatory factors in alveolar lavage fluid and plasma; preferably, said inflammatory factors include TNF-α, IL-1β, IFN-γ, IL-6, TGF-β1, IL-4, MCP-1/CCL2.

Further, wherein said drug is used to reduce levels of a plurality of pro-fibrotic factors and inflammatory cytokines and type I collagen in alveolar lavage fluid and/or plasma, and/or to increase levels of anti-fibrotic factors; preferably, said pro-fibrotic factors and inflammatory cytokines are TNF-α, IL-1β, IL-6, MCP-1, PDGF, SMAD3, SMAD2, MMP9. MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

Further, wherein said abnormalities of lipid metabolism comprise elevated levels of triglycerides and total cholesterol and/or reduced levels of leptin.

Further, wherein said dosage of the drug hydroxyprogesterone caproate is 30-100 mg/day, or 250-750 mg/week, and/or the hydroxyprogesterone caproate administration regimen is 1-7 weekly administrations, preferably 1-4 weekly administrations, more preferably 1-2 weekly administrations.

Further, wherein said drug hydroxyprogesterone caproate is dosed at a daily dose of 20-80 mg/kg; preferably 40-80 mg/kg; more preferably 40 or 80 mg/kg.

On the other hand, the present application provides applications of hydroxyprogesterone caproate, or a combination of hydroxyprogesterone caproate and glucocorticoids in the preparation of drugs that inhibit pro-fibrotic factors and inflammatory cytokines, and/or type I collagen; and/or increase the level of anti-fibrotic factors; or applications as inhibitors of pro-fibrotic factors and inflammatory cytokines, and/or applications as inhibitors of type I collagen, and/or applications as promoters of anti fibrosis factor promoters.

Further, wherein said glucocorticoid is dexamethasone.

Further, wherein said inflammatory factor is TNF-α, IL-1β, IFN-γ, IL-6, TGF-β1, IL-4 and/or MCP-1/CCL2.

Further, wherein said pro-fibrotic factor and inflammatory cytokine are TNF-α, IL-1β, IL-6, MCP-1, PDGF, SMAD3, SMAD2, MMP9, MMP2, CXCL-12 and/or TGF-β1, and said anti-fibrotic factor is IFN-γ and/or IL-4; more preferably, said pro-fibrotic factor is TNF-α, IL-β1, IL-6, PDGF, SMAD3, SMAD2, MMP2 and/or TGF-β1.

Further, wherein said pharmaceutical dosage form is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

On the other hand, the present application provides methods of inhibiting pro-fibrotic factors and inflammatory cytokines, and/or type I collagen; and/or increasing levels of anti-fibrotic factors, said methods comprising administering hydroxyprogesterone caproate.

Further, wherein said method does not comprise the step of administering a glucocorticoid.

Further, wherein said method comprises the step of administering a glucocorticoid.

Further, wherein said glucocorticoid is dexamethasone.

Further, wherein said inflammatory factor is TNF-α, IL-1β, IFN-γ, IL-6, TGF-β1, IL-4 and/or MCP-1/CCL2.

Further, wherein said pro-fibrotic factor and inflammatory cytokine are TNF-α, IL-1β, IL-6, MCP-1, PDGF, SMAD3, SMAD2, MMP9, MMP2, CXCL-12 and/or TGF-β1, and said anti-fibrotic factor is IFN-γ and/or IL-4; more preferably, said pro-fibrotic factor is TNF-α, IL-β1, IL-6, PDGF, SMAD3, SMAD2, MMP2 and/or TGF-β1.

Further, wherein the dosage form of hydroxyprogesterone caproate is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein the dose of hydroxyprogesterone caproate is 30-100 mg/day, or 250-750 mg/week.

Further, wherein the daily dose of hydroxyprogesterone caproate is 20-80 mg/kg; preferably 40-80 mg/kg; more preferably 40 or 80 mg/kg.

Further, wherein the hydroxyprogesterone caproate administration regimen is administered 1-7 times per week, preferably 1-4 times per week, more preferably 1-2 times per week.

On the other hand, the present application provides drugs for inhibiting pro-fibrotic factors and inflammatory cytokines, and/or type I collagen; and/or increasing the level of anti-fibrotic factors, characterized in that said drugs comprise hydroxyprogesterone caproate.

Further, wherein hydroxyprogesterone caproate is the sole active ingredient in said drug.

Further, wherein said drug further comprises a glucocorticoid.

Further, wherein said glucocorticoid is dexamethasone.

Further, wherein said inflammatory factor is TNF-α, IL-1β, IFN-γ, IL-6, TGF-β1, IL-4 and/or MCP-1/CCL2.

Further, wherein said pro-fibrotic factor and inflammatory cytokine are TNF-α, IL-1β, IL-6, MCP-1, PDGF, SMAD3, SMAD2, MMP9, MMP2, CXCL-12 and/or TGF-β1, and said anti-fibrotic factor is IFN-γ and/or IL-4; more preferably, said pro-fibrotic factor is TNF-α, IL-13, IL-6, PDGF, SMAD3, SMAD2, MMP2 and/or TGF-β1.

Further, wherein said pharmaceutical dosage form is an injection, an oral formulation or a topical formulation; preferably an injection, an oral formulation; particularly preferred is an injection.

Further, wherein the injectable formulations include injections, powdered injections; the oral formulations include tablets, capsules, oral liquids, throat or nasal sprays, and the topical formulations include ointments, sprays, and patches.

Further, wherein said dosage of the drug hydroxyprogesterone caproate is 30-100 mg/day, or 250-750 mg/week, and/or the hydroxyprogesterone caproate administration regimen is 1-7 weekly administrations, preferably 1-4 weekly administrations, more preferably 1-2 weekly administrations.

Further, wherein the daily dose of hydroxyprogesterone caproate is 20-80 mg/kg; preferably 40-80 mg/kg; more preferably 40 or 80 mg/kg.

Further, said drug further comprises pharmaceutically acceptable excipients, said pharmaceutically acceptable excipients including, but not limited to, solvents, co-solvents, stabilizers, dispersants, viscosity modifiers, antioxidants, sweeteners, binders, and gaseous generators.

Further, in said method or said drug, hydroxyprogesterone caproate is the sole active ingredient in the method or the drug; or in the method further comprising administering other active ingredients or the drug further comprising other active ingredients; said other active ingredients include, but are not limited to, glucocorticosteroid drugs, anti-inflammatory drugs other than glucocorticosteroid, anti-fibrotic drugs such as pirfenidone, nidanib, antacid drugs that Drugs or devices that increase blood oxygen levels.

On the other hand, the present application provides applications in the preparation of hydroxyprogesterone caproate for the reduction of glucocorticoid-induced abnormalities of lipid metabolism.

Further, wherein said glucocorticoid is dexamethasone.

Further, wherein said abnormalities of lipid metabolism comprise elevated levels of triglycerides and total cholesterol and/or reduced levels of leptin.

On the other hand, the present application provides methods of reducing glucocorticoid-induced abnormalities of lipid metabolism characterized by comprising administering hydroxyprogesterone caproate.

Further, wherein said glucocorticoid is dexamethasone.

Further, wherein said abnormalities of lipid metabolism comprise elevated levels of triglycerides and total cholesterol and/or reduced levels of leptin.

In the present application, the daily dose of hydroxyprogesterone caproate is 20-80 mg/kg; preferably 40-80 mg/kg; more preferably 40 or 80 mg/kg; and/or the dose of dexamethasone is 1-2 mg/kg, preferably 1 mg/kg or 2 mg/kg; wherein hydroxyprogesterone caproate and dexamethasone are administered in combination with the following preferred administration regimen: hydroxyprogesterone caproate 40 mg/kg+Dexamethasone 2 mg/kg, preferably hydroxyprogesterone caproate 40 mg/kg+dexamethasone 1 mg/kg.

Hydroxyprogesterone caproate is also referred to in this application as 17-alpha hydroxyprogesterone caproate, 17-HPC, 17-hydroxyprogesterone caproate, 17-hydroxyhexylprogesterone, CAS No. 630-56-8, and in the experimental section the designation PR2005 is used.

Interstitial pneumonias described in this application include, but are not limited to idiopathic interstitial pneumonia (idiopathic interstitial pneumonia, IIP), including various subtypes of idiopathic interstitial pneumonia including, but not limited to, idiopathic pulmonary fibrosis (IPF), nonspecific interstitial pneumonia (NSIP), cryptogenic organoid pneumonia (COP), acute interstitial pneumonia (AIP), respiratory bronchiolitis with interstitial lung disease (RBILD), desquamative interstitial pneumonia (DIP), and lymphocytic interstitial pneumonia (LIP); preferably idiopathic pulmonary fibrosis (IPF).

The pulmonary fibrosis described in the present application includes pulmonary fibrosis due to various causes, including but not limited to idiopathic pulmonary fibrosis, primary pulmonary fibrosis, secondary non-fibrotic, interstitial pulmonary fibrosis, and the like; idiopathic pulmonary fibrosis is preferred.

The drugs of the present application may be used alone or in combination with drugs and therapies known in the art for the treatment of interstitial pneumonia or for the relief of symptoms associated with interstitial pneumonia, which include, but are not limited to, glucocorticoid medications, anti-inflammatory drugs other than glucocorticosteroids, anti-fibrotic medications such as pirfenidone, nidanib, antacid medications, and medications or devices that increase blood oxygen levels.

The available dosage forms of the present application include, but are not limited to, injectable preparations such as injections, powders, etc., oral preparations such as tablets, capsules, oral liquids, etc., throat or nasal sprays, topical preparations such as ointments, sprays, patches, etc.; preferably injections, oral preparations; oral preparations are particularly preferred. The different drugs in the pharmaceutical composition may be prepared into the same formulation; they may also be provided in the same package in the same or different dosage forms; or in the same or different dosage forms in their respective separate packages.

A person skilled in the art may select a suitable excipient for the drug covered by the present application based on general knowledge in the field of pharmacy, and the types of excipients available include, but are not limited to, solvents, co-solvents, stabilizers, dispersing agents, viscosity modifiers, antioxidants, sweeteners, binders, gas-generating agents, and the like.

The experiments showed that hydroxyprogesterone caproate could significantly improve the changes of various inflammatory cytokines in the blood of rats in the bleomycin-induced disease model, and the combined effect was better than that of the positive control drug, pirfenidone; hydroxyprogesterone caproate showed a significant inhibitory effect on a variety of pro-fibrotic proteins and collagen type I COL I in the lung tissues of rats; hydroxyprogesterone caproate could improve the lipid metabolism abnormality induced by the dexamethasone treatment when it is used in conjunction with dexamethasone. The combination of hydroxyprogesterone caproate and dexamethasone improved the lipid metabolism induced by dexamethasone alone. The above results provide new drug types and development direction for the treatment of interstitial pneumonia.

### BRIEF DESCRIPTION OF THE DRAWINGS

FIG. 1 shows that 17-αhydroxyprogesterone significantly ameliorated bleomycin-induced changes in multiple inflammatory cytokines in the blood of rats (data are mean±standard error, N=6-10, * p<0.05, ** p<0.01, *** p<0.001 compared with the model group);

FIG. 2 shows that 17-αhydroxyprogesterone significantly ameliorated bleomycin-induced changes in multiple pro-fibrotic proteins as well as type I collagen in rat lung tissues (data are mean±standard error, N=3, compared with the model group * p<0.05, ** p<0.01, *** p<0.001);

FIG. 3 shows the effect of 17-αhydroxyprogesterone on lipid metabolism abnormalities induced by dexamethasone treatment in a bleomycin-induced rat model of pulmonary fibrosis (data are mean±standard error, N=6-10).

### DETAILED DESCRIPTION

### Primary Drugs and Reagents:

Hydroxyprogesterone Caproate: purchased from Symbiotec;

Bleomycin hydrochloride (BLM): purchased from HanHui Pharmaceutical Co., Ltd; (Bleomycin 15 mg added to 3 ml of saline to obtain a concentration of 5 mg/ml solution);

Dexamethasone Hydrochloride Tablets (DEX): purchased from Shanghai Jinbuyao Lankao Pharmaceutical Co;

Pirfenidone capsule (PFD): purchased from Beijing Contini Pharmaceutical Co.

### Example 1 Establishment of Rat Model

SD rats (weighing 240-280 g) were purchased from Beijing Viton Lihua Laboratory Animal Technology Co. Ltd (Animal License No.: SCXK (Beijing) 2016-0006; Animal Qualification Certificate No.: NO. 110011201110788686).

The quarantine and domestication period was 1 week, after which rats were serpentinely grouped by body weight into control and interstitial pneumonia model groups. On day 0 of the study, rats in the model group were given a single dose of 5 mg/kg bleomycin by a single intratracheal injection of bleomycin. The rats in the control group were given a single drop of saline. Immediately after completion, the animals were placed in an upright position and rotated to facilitate uniform distribution of the drug in the lungs. The animals were placed prone in a warm environment for natural awakening. Animals were fed and watered freely after awakening.

### Example 2 Experimental Grouping

| | mold-making | drug delivery |
|---|---|---|
| groups | (single-dose, day 0) | (once daily, days 1-28) |
| 1. sham treatment | saline (medicine) | saline (medicine) |
| control group | | |
| (n = 10) (Con) | | |
| 2. Model Group | Bleomycin (5 mg/kg) | saline (medicine) |
| (n = 10) (BLM) | | |
| 3. Low dose group | Bleomycin (5 mg/kg) | Hydroxyprogesterone |
| (n = 10) (EG20) | | caproate (20 mg/kg, by |
| | | gavage) |
| 4. Medium-dose | Bleomycin (5 mg/kg) | Hydroxyprogesterone |
| group (n = 10) | | caproate (40 mg/kg, |
| (EG40) | | gavage) |
| 5. High-dose group | Bleomycin (5 mg/kg) | Hydroxyprogesterone |
| (n = 10) (EG80) | | caproate (80 mg/kg, |
| | | gavage) |
| 6. Pirfenidone group | Bleomycin (5 mg/kg) | Pirfenidone (54 mg/kg) |
| (n = 10) (PFD) | | |
| 7. Dexamethasone | Bleomycin (5 mg/kg) | Dexamethasone |
| group (n = 10) | | (2 mg/kg, gavage) |
| (DEX2) | | |
| 8. Combination | Bleomycin (5 mg/kg) | Hydroxyprogesterone |
| group (n = 10) | | caproate |
| (EG40 + DEX2) | | (40 mg/kg, gavage) + |
| | | Dexamethasone |
| | | (2 mg/kg, gavage) |
| 9. Combination | Bleomycin (5 mg/kg) | Hydroxyprogesterone |
| group (n = 10) | | caproate |
| (EG40 + DEX2) | | (40 mg/kg, gavage) + |
| | | dexamethasone |
| | | (1 mg/kg, gavage) |

### Example 3 Indicators Tested

### General Observation and Examination:

Body weight, appearance and signs, coat condition, behavioral activities, the presence of tremors, spasms and other neurological reactions, the presence of abnormal breath sounds and abnormal gait and movement, the response to sound fright, the shape and color of feces, and so on. Observe the lower abdomen and anus for dirt and excrement, the responsiveness when catching, the state of the eyes, nose and mouth, and the state of the skin (with or without color change, trauma, tumor, etc.) when administering the drug.

### Alveolar Lavage Fluid Collection

After the last administration, rats in each group were fasted without water for 12 h, anesthetized by intraperitoneal injection of 100 mg/L chloral hydrate (3.5 mL/kg), the muscles were separated, the trachea was exposed, a T-shaped opening was cut in the trachea, a tracheal syringe was inserted to inject 2 ml of PBS into the lung tissues, which was then gently sucked out, and then centrifuged at 2500 r/min for 10 min, and the supernatant was aspirated, which was used for the detection of cytokine content. The supernatant was centrifuged at 2500 r/min for 10 min and the supernatant was aspirated for the detection of cytokine levels, while the cellular precipitate was collected for the detection of inflammatory cells.

### Categorical Count of Inflammatory Cells in Alveolar Lavage Fluid

The cell precipitate in alveolar lavage fluid was taken, added dropwise to the slide, air-dried, Rhesus-Giemsa Stain A was added, fixed for 2 min at room temperature, then Rhesus-Giemsa Stain B was added and equilibrated for 10 min, washed twice with double-distilled water, air-dried, and sorted and counted for the number of leukocytes, the number of neutrophils, the number of eosinophils, the number of lymphocytes, and the number of macrophages.

### Cytokine Detection

After the last administration, rats in each group were fasted without water for 12 h, anesthetized by intraperitoneal injection of 100 mg/L chloral hydrate (3.5 mL/kg), blood was taken from the abdominal aorta, and the serum was separated by centrifugation at 3500 rpm for 15 min. ELISA was performed to detect the cytokines TNF-α, IL-1β, IFN-γ, IL-6, TGF-β1, and IL-4 in alveolar lavage fluid and serum, MCP-1/CCL2, and to detect the content of HYP, MMP2, MMP9, αvβ6, and VEGF in lung tissues, the steps were the same as the standard procedure of the kit.

### Dexamethasone Side Effect Indicator Test

Serum potassium, glucose, triglycerides (TGs), total cholesterol (TC), free fatty acid (FFA), high density lipoprotein (HDL-c), low density lipoprotein (LDL-c), corticosterone (Corticosterone), and blood glucose were measured. HDL-c), low-density lipoprotein cholesterol (LDL-c), corticosterone (CORT), insulin, leptin, 5-hydroxy tryptamine (5-HT), 5-hydroxytryptamine (5-HT), and 5-HT. 5-hydroxy tryptamine (5-HT) and norepinephrine (NE).

### Example 4 Experimental Results

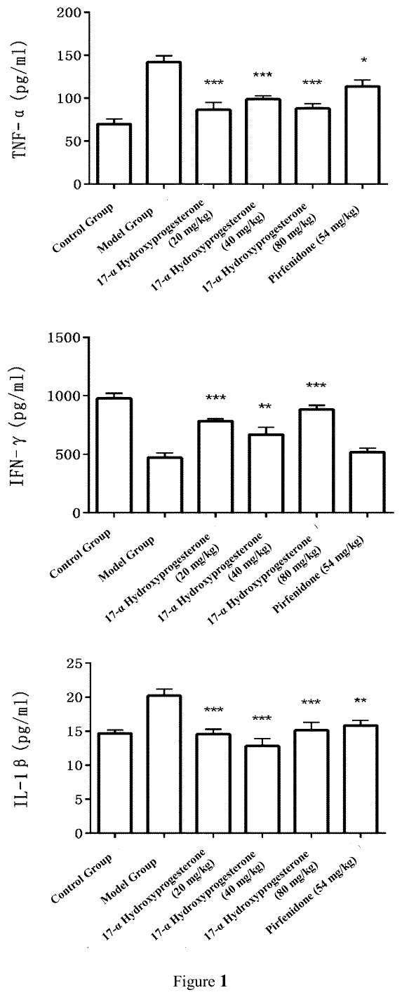

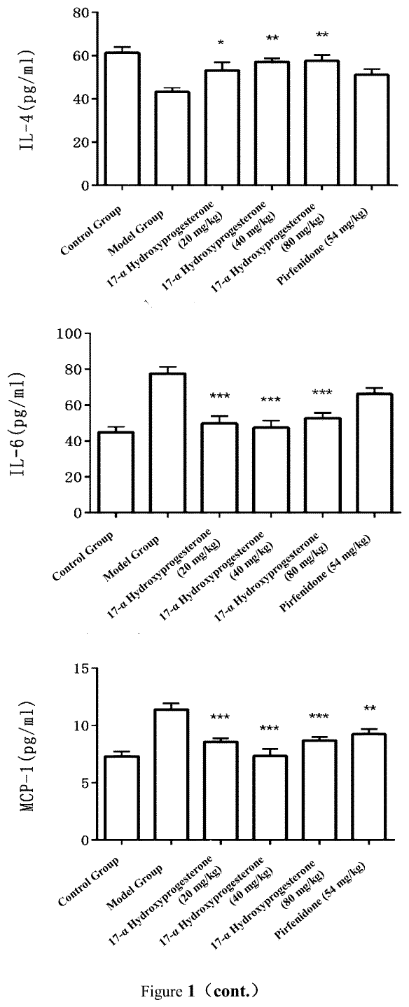

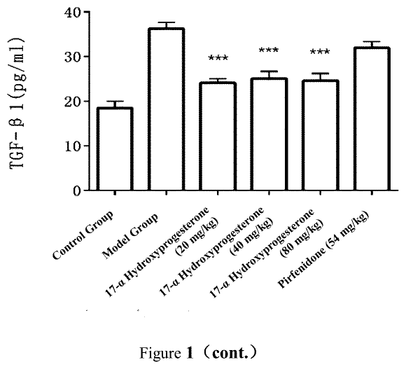

(1) In the bleomycin-induced rat model of pulmonary fibrosis (FIG. 1), analysis of blood samples showed that bleomycin induced a significant increase in blood levels of inflammatory and pro-fibrotic factors, including TNF-α, IL-1β, IL-6, MCP-1, and TGF-β1, and that treatment with hydroxyprogesterone caproate significantly reduced the levels of these factors compared to normal control rats, and that this was statistically significance. In addition, bleomycin decreased the blood levels of IFN-γ and IL-4, and 17-alpha hydroxyprogesterone treatment significantly and statistically increased the levels of these two factors compared to normal control rats. The combined improvement effect of hydroxyprogesterone caproate on inflammatory and pro-fibrotic factors in the blood was significantly better than that of the positive control drug pirfenidone.

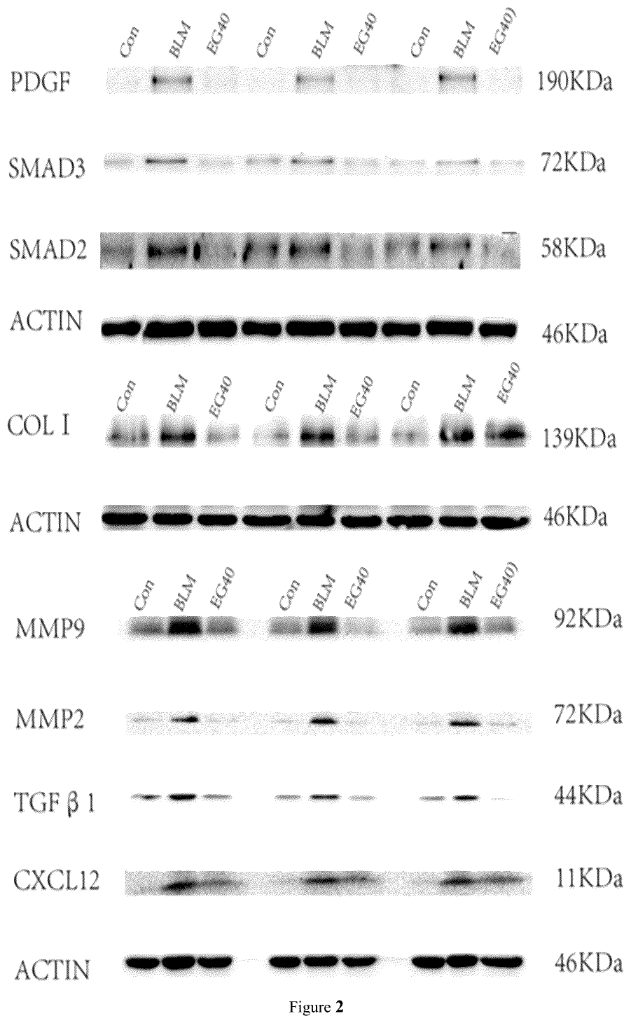

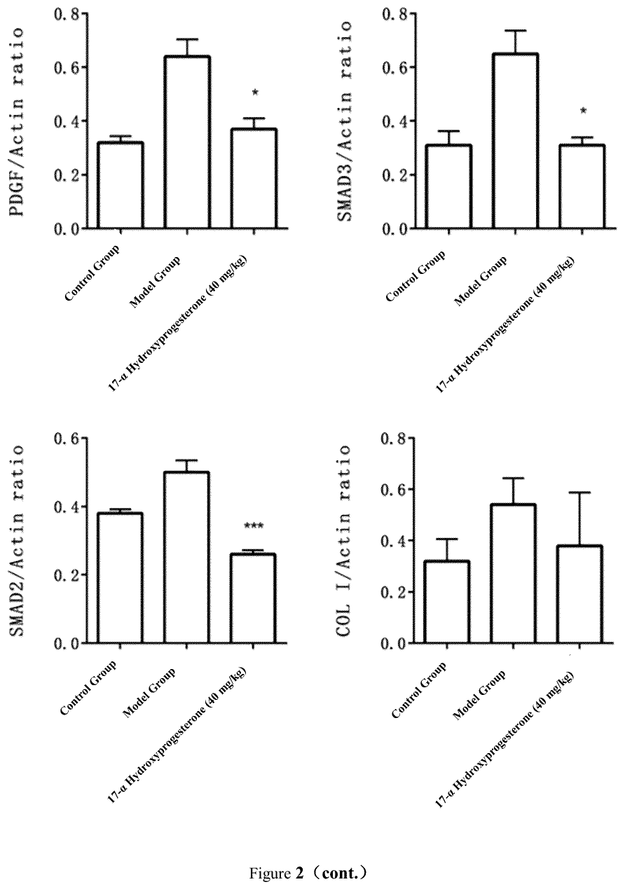

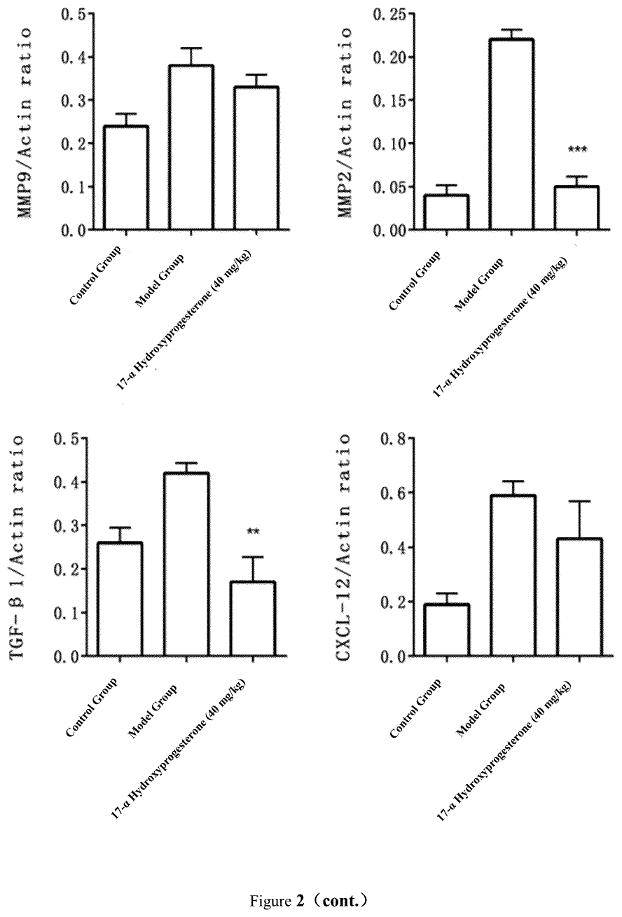

(2) In the bleomycin-induced pulmonary fibrosis model in rats, further analysis of rat lung tissues showed (FIG. 2) that bleomycin induced an increase in the levels of a number of pro-fibrotic factors, including PDGF, SMAD3, SMAD2, MMP9, MMP2, CXCL-12, TGF-β1, and collagen type I COL I proteins, in lung tissues, when compared to normal control rats. Hydroxyprogesterone caproate treatment, on the other hand, reduced the levels of these proteins, with statistically significant changes in PDGF, SMAD3, SMAD2, MMP2, and TGF-β1.

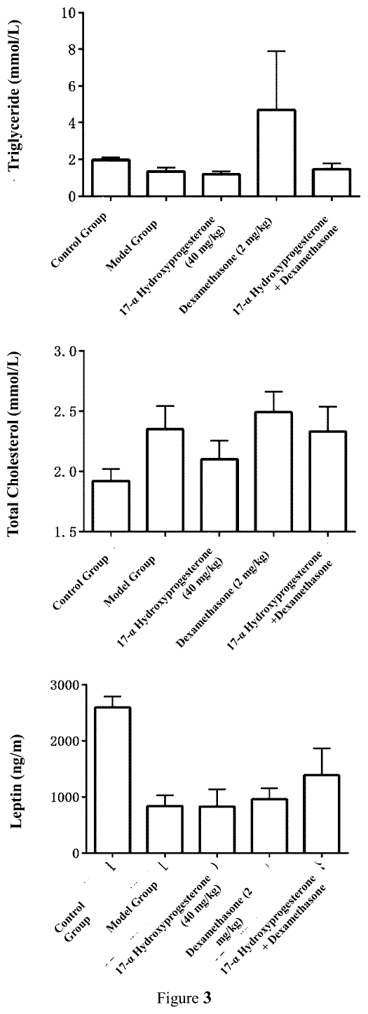

(3) In the bleomycin-induced pulmonary fibrosis model in rats, analysis of the indicators related to lipid metabolism in the blood of rats showed (FIG. 3) that dexamethasone treatment caused an increase in triglycerides and total cholesterol in the blood, with the increase in triglycerides being more pronounced. The combination of hydroxyprogesterone caproate and dexamethasone reduced blood triglyceride and total cholesterol levels compared to the dexamethasone treatment group. In addition, in this disease model, leptin levels were significantly reduced in the model group, the hydroxyprogesterone caproate group, and the dexamethasone group compared with the normal control rats, and the combination of hydroxyprogesterone caproate and dexamethasone showed a tendency to increase the leptin levels in the blood.

In summary, in the bleomycin-induced rat lung fibrosis model, hydroxyprogesterone caproate can significantly improve the changes of multiple inflammatory cytokines in the blood of rats, and the combined effect is better than that of the positive control drug, pirfenidone; hydroxyprogesterone caproate showed significant inhibitory effects on a variety of pro-fibrotic proteins and collagen type I COLI in the rat lung tissues; hydroxyprogesterone caproate can improve the lipid metabolism abnormality induced by dexamethasone when used in conjunction with dexamethasone. Hydroxyprogesterone caproate in combination with dexamethasone can improve lipid metabolism induced by dexamethasone alone.

In addition, two pathogenetic features, inflammation and pulmonary fibrosis, were reflected in the bleomycin-induced pulmonary fibrosis model in rats. And these two features are also the main manifestations of idiopathic interstitial pneumonia. From the above effects of hydroxyprogesterone caproate or the combination of hydroxyprogesterone caproate and dexamethasone in the bleomycin-induced rat model of pulmonary fibrosis, it can be fully expected that it is also efficacious in idiopathic interstitial pneumonia.

1.-14. (canceled)

15. Method of treating pulmonary disease, wherein the method comprises administering hydroxyprogesterone caproate to pulmonary disease patients, wherein the treating pulmonary disease comprises (a) treating interstitial pneumonia; (b) treating pulmonary fibrosis or (c) inhibiting pro-fibrotic factors and inflammatory cytokines, and/or type I collagen; and/or increasing levels of anti-fibrotic factors.

16. A method according to claim 15, wherein said method does not comprise the step of administering a glucocorticoid.

17. A method according to claim 15, wherein said method comprises the step of administering a glucocorticoid.

18. The method according to claim 17, wherein said glucocorticoid is dexamethasone.

19. The method according to claim 15, wherein the dosage form of hydroxyprogesterone caproate is an injectable, oral or topical formulation; preferably an injectable, oral formulation; particularly preferred is an injectable formulation.

20. The method according to claim 19, wherein the injectable formulations include injectable solutions, powdered injections; oral formulations include tablets, capsules, oral liquids, throat or nasal sprays, and topical formulations include ointments, sprays, and patches.

21. The method according to claim 15, wherein the dose of hydroxyprogesterone caproate is 30-100 mg/day, or 250-750 mg/week.

22. The method according to claim 15, wherein the daily dose of hydroxyprogesterone caproate is 20-80 mg/kg; preferably 40-80 mg/kg; more preferably 40 or 80 mg/kg.

23. The method according to claim 15, wherein the hydroxyprogesterone caproate administration regimen is administered 1-7 times per week, preferably 1-4 times, more preferably 1-2 times.

24. The method according to claim 15, wherein said treating pulmonary disease comprises reducing symptoms of inflammation and fibrosis in the lungs.

25. A method according to claim 15, wherein said treating pulmonary disease comprises reducing collagen deposition in the lungs.

26. The method according to claim 15, wherein said treating pulmonary disease comprises reducing inflammatory cell counts in bronchoalveolar lavage fluid.

27. A method according to claim 15, wherein said treating pulmonary disease comprises reducing hydroxyproline levels in lung tissue.

28. The method according to claim 15, wherein said treating pulmonary disease comprises reducing the levels of a plurality of inflammatory factors in alveolar lavage fluid and plasma; preferably, said inflammatory factors comprise TNF-α, IL-1β, IFN-γ, IL-6, TGF-β1, IL-4, MCP-1/CCL2.

29. The method according to claim 15, wherein said treating pulmonary disease comprises reducing the levels of a plurality of pro-fibrotic and inflammatory cytokines and type I collagen in alveolar lavage fluid and/or plasma, and/or increasing the levels of an anti-fibrotic factor; preferably said pro-fibrotic factors and inflammatory cytokines are TNF-α, IL-1β, IL-6, MCP-1, PDGF, SMAD3, SMAD2, MMP9, MMP2, CXCL-12 and/or TGF-β1, and said anti-fibrotic factor is IFN-γ and/or IL-4; more preferably, said pro-fibrotic factor is TNF-α, IL-1β, IL-6, PDGF, SMAD3, SMAD2, MMP2 and/or TGF-β1.

30. The method according to claim 17, wherein said treatment of abnormalities of lipid metabolism during pulmonary disease is reduced.

31. The method according to claim 30, wherein said abnormalities of lipid metabolism comprise elevated levels of triglycerides and total cholesterol and/or reduced levels of leptin.

32. A drug for treating pulmonary disease wherein said drug comprises hydroxyprogesterone caproate, wherein the treating pulmonary disease comprises (a) treating interstitial pneumonia; (b) treating pulmonary fibrosis or (c) inhibiting pro-fibrotic factors and inflammatory cytokines, and/or type I collagen; and/or increasing levels of anti-fibrotic factors.

33.-122. (canceled)
